(19) United States
(12) Patent Application Publication
(10) Pub. No.: US 2002/0077651 A1
(43) Pub. Date: Jun. 20, 2002

(54) Side branch dilatation catheter

(76) Inventors: David R. Holmes JR., Rochester, MN (US); Robert S. Schwartz, Rochester, MN (US)

Correspondence Address: JOHN F. HAYDEN, Fish & Richardson P.C., 601 Thirteenth Street, NW, Washington, DC, 20005, US

(21) Appl. No.: 09/736,276

(22) Filed: Dec. 15, 2000

(51) Int. Cl. A61B 17/00

(52) U.S. Cl. 606/190

(57) ABSTRACT

A system for delivering a guide wire to an artery and a side branch vessel of the artery includes a delivery catheter and a pair of guide wires. The delivery catheter includes a first lumen with a first opening and a second lumen with a second opening. The first guide wire is configured to extend through the first lumen and the second guide wire is configured to extend through the second lumen. The first opening is configured to direct the first guide wire into the side branch vessel, and the second opening is configured to direct the second guide wire into the main artery.

### TECHNICAL FIELD

This invention relates to a catheter-based system for delivering a balloon catheter to dilate an artery or to place a catheter or tube in a vessel.

### BACKGROUND

Coronary artery disease involves a buildup of biological material, typically referred to as plaque, within the lumen of a coronary artery. The site of the plaque buildup is referred to as a lesion. As the buildup progresses, it reduces the diameter of the lumen through which blood can flow. If the buildup completely or sufficiently reduces the diameter, distal coronary tissue will not receive a sufficient supply of oxygenated blood, which is referred to as myocardial ischemia. If the diameter is completely blocked, the condition is referred to as a myocardial infarction, more commonly known as a heart attack.

Cardiologists address the buildup of plaque in an artery by a number of interventional procedures, including atherectomy, angioplasty, and stenting. After obtaining arterial access, a guide wire is advanced into a coronary artery that has a buildup of plaque. An inflatable balloon catheter may be passed over the guide wire, advanced into the lesion, and inflated to increase the diameter of the lumen or deliver a stent. The wire and inflatable balloon catheter are then withdrawn from the artery. However, coronary artery disease typically is not limited to one lesion in one artery and therefore the cardiologist may need to repeat the catheterization and balloon inflation or stent procedures in other arteries to open additional lesions. Additional lesions often involve arterial side branch vessels that may be narrowed causing symptoms by on their own. Access to these side branch vessels can be difficult.

### SUMMARY

In one general aspect, a system for delivering a guide wire to an artery and a side branch vessel of the artery includes a delivery catheter and a pair of guide wires. The delivery catheter includes a first lumen with a first opening and a second lumen with a second opening. The first guide wire is configured to extend through the first lumen and the second guide wire is configured to extend through the second lumen. The first opening is configured to direct the first guide wire into the side branch vessel, and the second opening is configured to direct the second guide wire into the main artery.

Embodiments of the system may include one or more of the following features. For example, the catheter may further include a radiopaque indicator adjacent to the first opening. The radiopaque indicator may include at least one stud attached to the shaft. The radiopaque indicator also may include a ring that encircles at least a part of the circumference of the shaft. The ring may include an opening and the opening is adjacent to the first opening in the shaft. The opening in the ring also may be coaxial with the first opening in the shaft. The ring may include an extension that projects into the first opening in the shaft. The ring may include a pair of bands that are joined at a first point of the circumference of the bands and are separated at a second point of the circumference of the bands, and the first opening is positioned between a separation at the second point of the pair of bands. The ring may be designed to include a cut-out section.

The catheter may include a first section including the first lumen and the second lumen and extending between the first end and the first opening, and a second section including the second lumen and extending between the first opening and the second end, and the second section has a smaller cross-section than a cross-section of the first section. The catheter may include an aimer positioned at least in part on the second section in a position that is adjacent to the first section. The aimer may include a surface that is configured to direct the guide wire in the first lumen in a direction away from the catheter when the guide wire passes through the first opening.

The catheter may include an inflatable balloon positioned on the catheter adjacent to the first opening such that a guide wire passing through the first opening is deflected by the balloon. The system may further include at least one balloon inflation catheter configured to be delivered over one of the guide wires.

In another general aspect, a delivery catheter is configured to deliver a first guide wire to an artery and a second guide wire to a side branch vessel of the artery. The delivery catheter includes a shaft having a first end and a second end, a first lumen in the shaft passing to a first opening in the shaft, and a second lumen passing to a second opening in the shaft.

Embodiments of the delivery catheter may include one or more of the following features. For example, the catheter may include a radiopaque indicator and the radiopaque indicator may include at least one stud attached to the shaft. The radiopaque indicator also may include a ring that encircles at least a part of the circumference of the shaft. The ring may include an opening that is adjacent to the first opening in the shaft or coaxial with the first opening in the shaft. The ring also may include an extension that projects into the first opening in the shaft. The ring also may include a pair of bands that are joined at a first point of the circumference of the bands and are separated at a second point of the circumference of the bands, and the first opening is positioned between a separation at the second point of the pair of bands. The ring also may include a cut-out section.

The shaft may include a first section including the first lumen and the second lumen and extending between the first end and the first opening, and a second section including the second lumen and extending between the first opening and the second end, and the second section has a smaller cross-section than a cross-section of the first section. The shaft may include an aimer positioned at least in part on the second section in a position that is adjacent to the first section. The aimer may include a surface that is configured to direct the guide wire in the first lumen in a direction away from the catheter when the guide wire passes through the first opening.

The catheter may include an inflatable balloon positioned on the catheter adjacent to the first opening such that a guide wire passing through the first opening is deflected by the balloon.

In another general aspect, a method of catheterizing an artery and a side branch of the artery includes providing a side branch delivery catheter that includes a shaft having a first end and a second end, a first lumen in the shaft passing to a first opening in the shaft, and a second lumen passing to a second opening in the shaft. The delivery catheter then is inserted into the vasculature and advanced into an artery. The delivery catheter is positioned such that the first opening is adjacent to the side branch of the artery. A first guide wire is inserted into the opening in the hub and advanced into the first lumen. A second guide wire is inserted into the opening in the hub and advanced into the second lumen. The first guide wire is advanced through the first opening and into the side branch of the artery.

Embodiments of the method may include one or more of the following features. For example, the method may further include advancing the second guide wire through the second opening and into the artery. The method also include providing an inflatable balloon catheter having an inflatable balloon, advancing the inflatable balloon catheter over the first guide wire, and deploying the inflatable balloon in the side branch of the artery. The method also may include providing an inflatable balloon catheter having an inflatable balloon, advancing the inflatable balloon catheter over the second guide wire, and deploying the inflatable balloon in the artery.

In the method, positioning the first opening adjacent to the side branch of the artery includes viewing a radiopaque indicator mounted on the catheter under fluoroscopy to orient the indicator relative to the side branch of the artery.

The side branch delivery catheter can provide numerous advantages. For example, the catheter permits the cardiologist to deploy a first guide wire into a coronary artery and a second guide wire into a side branch vessel without needing to exchange catheters to deploy the second guide wire. In this manner, the procedure can be accomplished in a single intervention, in a shorter period of time, and with reduced incidence of intra-vessel trauma. The catheter can facilitate the placement of stents or inflatable balloons to treat both an artery and a side branch extending from that artery. It also can facilitate the delivery of balloons or other treatment devices into branch vessels around severe bends or in vessels with severe tortuousity. It can facilitate the delivery of drugs or chemicals to a branch vessel, e.g., for infusion into the septal artery for treatment of hypertrophic cardiomyopathy. The catheter also can facilitate procedures such as stent delivery, drug delivery, gene therapy, ablation therapy, and electro-physiology therapy. The catheter also can be used to deliver pacing leads for left ventricular pacing and congestive heart failure pacing.

The details of one or more embodiments of the invention are set forth in the accompanying drawings and the description below. Other features and advantages of the invention will be apparent from the description and drawings, and from the claims.

### DESCRIPTION OF DRAWINGS

FIG. 1 is a side view of a side branch delivery catheter.

FIG. 2 is a side view of a guide wire.

FIG. 3 is a side view of an inflatable balloon catheter.

FIG. 4 is a side view of the side branch delivery catheter and the guide wire in an artery with a side branch. FIG. 5 is a side view of the side branch delivery catheter of FIG. 1 using hidden view lines to show the lumens.

FIG. 6 is an end view of a hub of the side branch delivery catheter of FIG. 1.

FIG. 7 is a side view of a radiopaque band adjacent to a lateral side opening of the side branch delivery catheter of FIG. 1.

FIG. 8 is a side view of a pair of radiopaque studs adjacent to a lateral side opening of the side branch delivery catheter of FIG. 1.

FIG. 9 is a side view of a radiopaque band having an opening positioned adjacent to a lateral side opening of the side branch delivery catheter of FIG. 1.

FIG. 10 is a side view of a radiopaque band having an extension projecting into a lateral side opening of the side branch delivery catheter of FIG. 1.

FIG. 11 is a side view of the band of FIG. 10.

FIG. 12 is a side view of a band having an extension and a semi-circular portion.

FIG. 13 is a perspective view of a ring having a pair of diverging bands.

FIG. 14 is a side view of a side branch delivery catheter having a guide wire aimer.

FIGS. 15 and 16 are side and end views, respectively, of the guide wire aimer of FIG. 14.

FIG. 17 is a side view of a side branch delivery catheter having an inflatable balloon.

FIG. 18 is a side view of the side branch delivery catheter of FIG. 17 deployed in a coronary artery.

FIG. 19 is a side view of a side branch delivery catheter having a pair of inflatable balloons adjacent to and on opposite sides of a side branch opening.

Like reference symbols in the various drawings indicate like elements.

### DETAILED DESCRIPTION

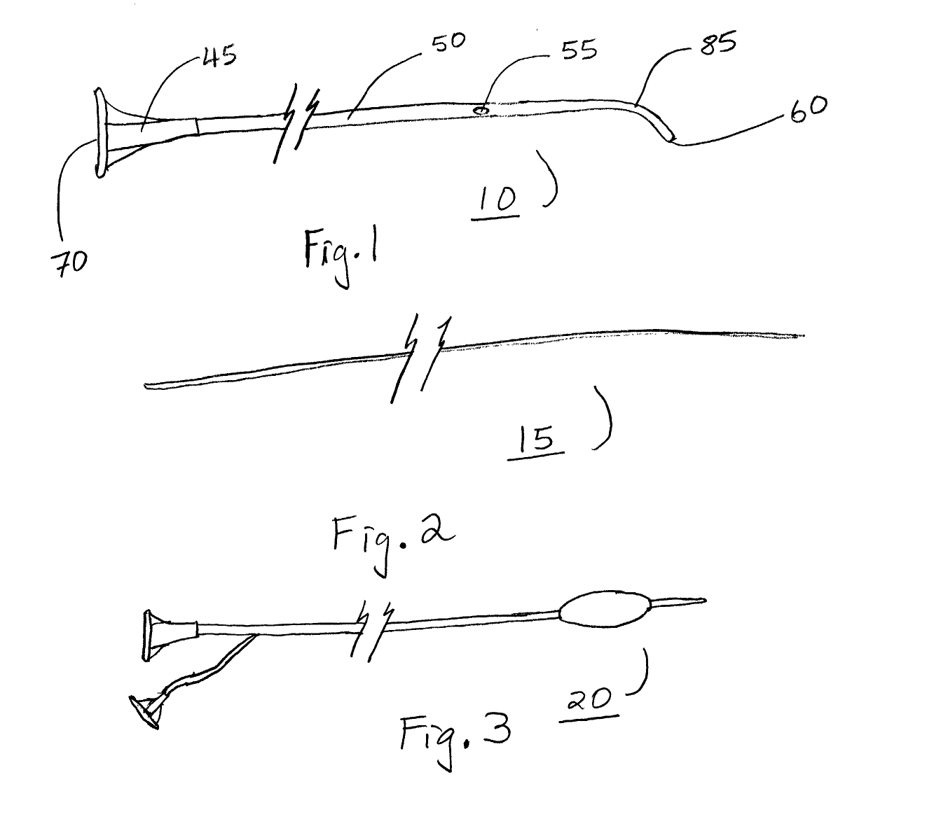

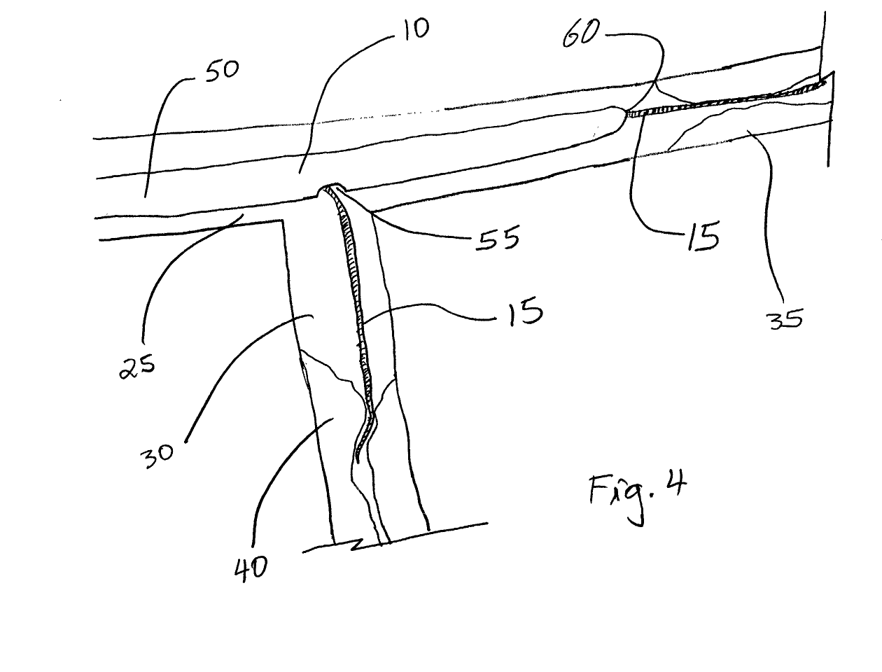

Referring to FIGS. 1-4 a catheter-based set of medical devices for side branch dilatation includes a side branch delivery catheter 10, one or more guide wires 15, and an inflatable balloon catheter 20. As described in more detail below, the catheters 10 and 20, and the guide wires 15 can be delivered into a coronary artery 25 and a side branch vessel 30 of the coronary artery 25 to treat a coronary artery lesion 35 and a side branch lesion 40.

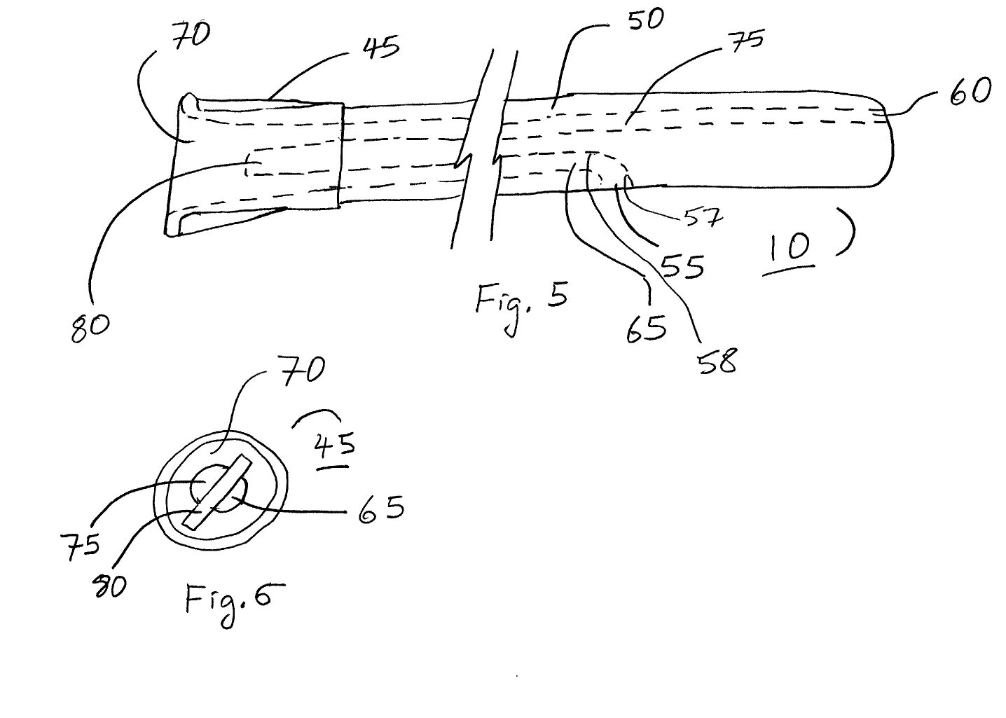

Referring also to FIGS. 5 and 6, the side branch delivery catheter 10 includes a hub 45, a shaft 50, a first or lateral opening 55, and a second or longitudinal opening 60. A first lumen 65 passes between a hub opening 70 and the lateral opening 55. A second lumen 75 passes between the hub opening 70 and the longitudinal opening 60. A divider 80 in the hub opening 70 directs guide wires 15 into the different lumens 65, 75. A curved region 85 of the delivery catheter 10 may be curved into any one of the many known curve shapes, such as one of the Judkin's Lefts or Rights, to ease the delivery of the catheter into a specific coronary artery.

The second opening includes a surface 57 that terminates at the circumference of the opening 55. Thus, when passing the guide wire 15 through the first lumen 65, the guide wire will be directed by the surface 57 out of and away from the catheter 10. The surface may be formed, for example, by using a tool with a hot surface to soften the material of the shaft 50 and press against the opening 55 to force material against an opposite side 58 of the lumen 65.

The delivery catheter 10 may be formed from any flexible, biocompatible material, such as for example nylon, polyethylene, polyurethane or a combination of those materials. Any of the other well-known polymers used to fabricate catheters also can be used. The shaft also can be braided using a wire braid or a synthetic fiber, such as Kevlar, as is well-known in the catheter art.

In use, the delivery catheter 10 first is advanced into an opening in, for example, the femoral artery, with or without the guide wires 15 being pre-placed within the lumens 65, 75. The catheter 10 is further advanced until the lateral opening 55 is adjacent to the side branch 30 and the guide wire 15 can be passed through the opening 55 into the side branch. To detect the side branch 30, a contrast dye injector (not shown) can be connected to the hub 45 and contrast dye injected to visualize the side branch and the coronary artery lesion 35.

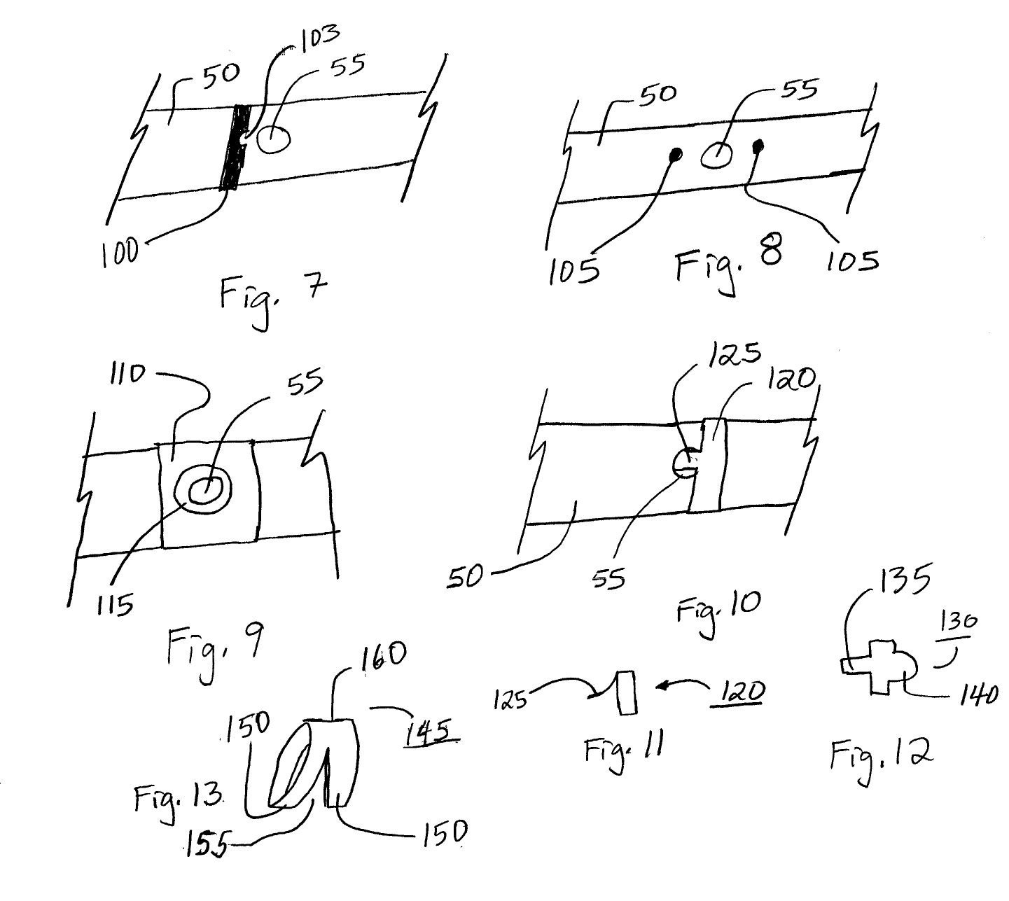

Referring to FIGS. 7-12, the delivery catheter 10 can be configured with a ring, band, stud, or other marker or indicator to help the cardiologist to visualize the lateral opening 55 during fluoroscopy. For example, referring to FIG. 7, a ring 100 encircles the circumference of the shaft 50 of the delivery catheter 10 at a position that is proximal to the opening 55. Of course, the ring 100 also may be placed at a position that is distal to the opening 55. An optional cut-out section 103 of the ring 100 is positioned adjacent to the opening 55 and may be used to orient the opening 55 under fluoroscopy. The ring 100 may be made of any radiopaque material that also is biocompatible, such as gold, and adhered to the shaft 50 by, for example, an adhesive, or by bending the ring around the circumference of the shaft so that it fits tightly around the shaft. The optional cut-out section 103 can be formed by slicing, cutting, or otherwise removing material from the ring 100. To maintain a smooth outer surface of the shaft 50, an indentation may be made around the outer circumference of the shaft and the ring 100 recessed into the indentation. When the cardiologist deploys the catheter 10 in the coronary artery 25 and visualizes the vasculature using fluoroscopy, the cardiologist is able to see the position of the ring 100 and place it adjacent to the side branch 30. The cardiologist then can advance the guide wire 15 through the opening 55 into the side branch.

Referring to FIG. 8, a pair of studs 105 may be embedded in the shaft 50 of the catheter 10; one stud being embedded at a distal position relative to the opening 55 and the other stud being embedded at a proximal position relative to the opening 55. Similarly to the ring 100, the studs 105 may be made of a radiopaque, biocompatible material, and adhered to the shaft 50. To maintain a smooth outer surface of the shaft 50, a pair of indentations may be made into the shaft and the studs 105 recessed into the indentations. The cardiologist can view the orientation of the studs 105 under fluoroscopy and torque and advance the catheter 10 until the positioning of the studs indicates that the lateral opening 55 is adjacent to the side branch 30. The cardiologist then can advance the guide wire 15 into the side branch 30.

Referring to FIG. 9, a ring 110 having an opening 115 is positioned on the shaft 50 such that the opening 115 is adjacent to and surrounds the lateral opening 55 such that the openings 115 and 55 are coaxial. The ring 110 may be made of the same material as the ring 100, adhered or attached to the shaft 50 in the same manner, and used under fluoroscopy in the same manner to direct the guide wire 15 into the side branch 30.

Referring to FIGS. 10 and 11, a ring 120 includes an extension 125 and is placed around the shaft 50 of the delivery catheter 10. The ring 120 is placed distally of the opening 55 with the extension 125 projecting into the opening 55 and the lumen 65. The extension 125 is curved such that it directs the guide wire 15 out of the lumen 65 through the opening 55 into the side branch 30. The ring 120 may be configured such that it does not form a complete circumference around the shaft 50 whereby the extension 125 can be inserted into the opening 55 and then the ring closed around the shaft 50. Referring also to FIG. 12, in a variation of the ring 120, a ring 130 includes an extension 135 and a semi-circular portion 140. The extension 135 projects into the opening 55 to direct the guide wire 15 into the side branch 30. The extension 135 and the semi-circular portion 140 orient the opening 55 under fluoroscopy so that the cardiologist can position the opening adjacent to the side branch vessel 30.

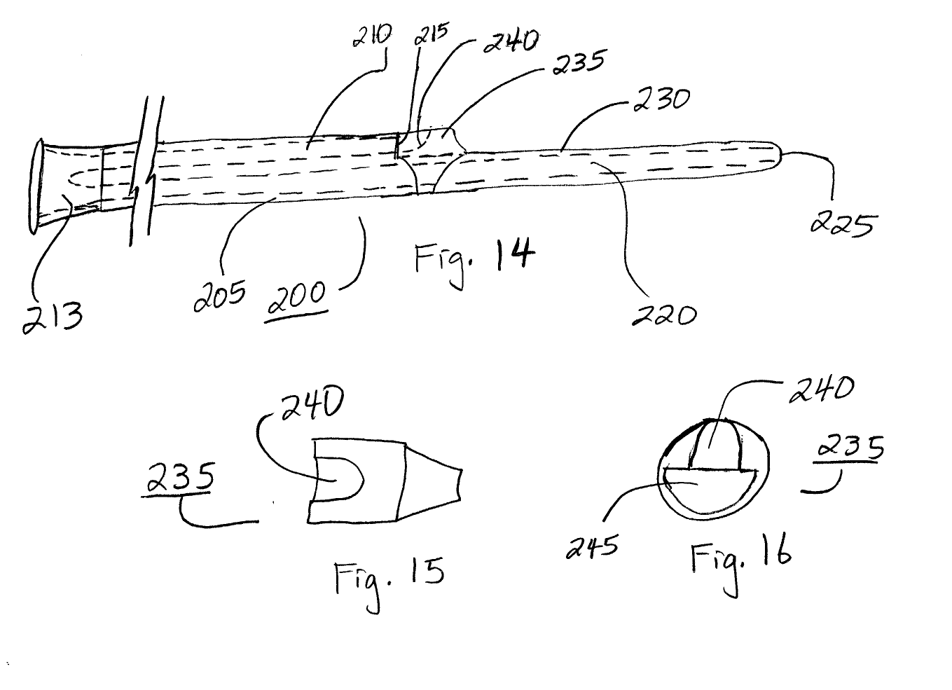

Referring to FIG. 13, a ring 145 includes a pair of circumferential bands 150 that surround the shaft 50. The bands 150 are maximally separated at one portion to form an opening 155 and are joined at an opposite portion to form a solid length 160 of the ring 145. The ring 145 is placed around the shaft 50 such that the opening 155 is adjacent to the lateral opening 55. Under fluoroscopy, the cardiologist is able to orient the opening 155 to place it adjacent to the side branch 30.

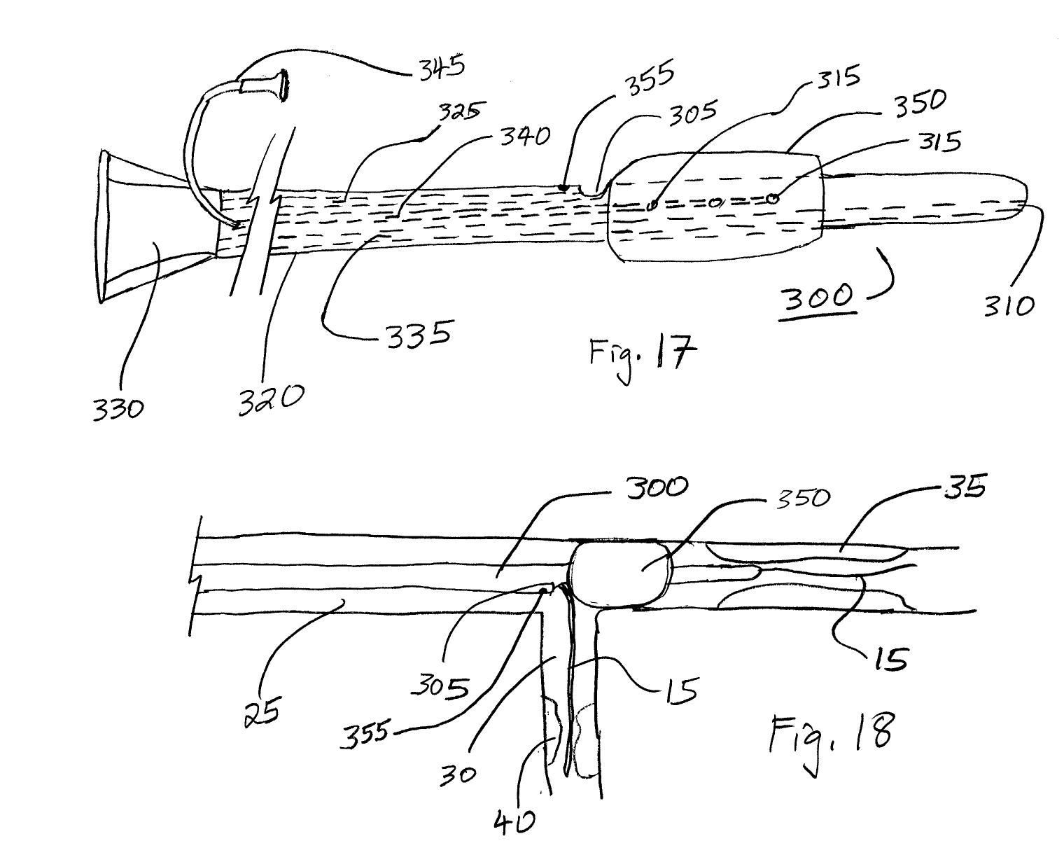

Referring to FIGS. 14-16, a side branch delivery catheter 200 includes a shaft 205 that includes a lateral-opening lumen 210 that passes between a hub 213 and a lateral opening 215. The catheter 200 also includes a longitudinal-opening lumen 220 that passes through the shaft 205 between the hub 213 and a longitudinal opening 225. A distal portion of the shaft 205 is removed to leave a smaller diameter section 230. The section 230 will be more compliant because there is less material in the shaft. A guide wire aimer 235 is positioned at the proximal end of the smaller diameter section 230 and is adjacent to the lateral opening 215. The aimer 235 includes a ramp 240 against which the guide wire 15 is directed to advance the guide wire 15 into the side branch vessel 30. The aimer 235 includes an opening 245 that is configured to be passed over the smaller diameter section 230 to mount the aimer to the shaft 205. The configuration of the aimer 235 may be varied. For example, the angle of the ramp 240 can be varied to vary an angle of projection of the guide wire 15 away from the catheter 200.

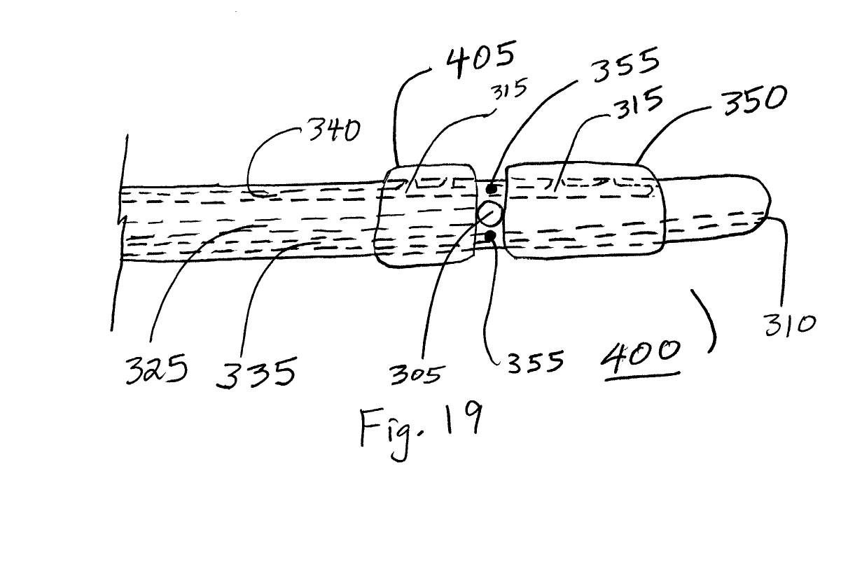

Referring to FIG. 17, a side branch delivery catheter 300 includes a lateral opening 305, a longitudinal opening 310, and inflatable balloon openings 315. The catheter 300 is formed from a shaft 320 having a lateral-opening lumen 325 that passes between a hub 330 and the lateral opening 305. The shaft 320 also includes a longitudinal-opening lumen 335 that passes between the hub 330 and the longitudinal opening 310, and a balloon lumen 340 that passes between a balloon inflation port 345 and balloon openings 315. A balloon inflation device (not shown) may be connected to the inflation port 345 to inflate a balloon 350. The inflatable balloon 350 is positioned distally of the lateral opening 305 and a radiopaque stud 355 is positioned proximally of the lateral opening. The stud 355 is positioned on the catheter 300 in a position adjacent to the opening 305 so that the cardiologist can orient the opening under fluoroscopy to be directed at the side branch vessel 30. The stud 355 can be replaced by other indicators or markers, as described above, that allow the cardiologist to orient the opening 305 relative to the side branch 30.

Referring also to FIG. 18, in use, the catheter 300 is inserted into a femoral artery and advanced into a coronary artery 25. Using the stud 355 or other similar orientation devices the catheter 300 is oriented with the opening 305 adjacent to the side branch vessel 30 of the coronary artery 25. The injector device is connected to the inflation port 345 and used to inflate the balloon 350. The first guide wire 15 then is passed through the longitudinal opening 355 and through the lesion 35. The second guide wire 15 then is passed through the lateral opening 305 and deflected by the balloon 350 into the side branch vessel 30.

A number of embodiments of the invention have been described. Nevertheless, it will be understood that various modifications may be made without departing from the spirit and scope of the invention. For example, referring to FIG. 19, side branch delivery catheter 400, which is a variation of the catheter 300, includes a second inflatable balloon 405 and a second stud 355 adjacent to the lateral opening 305. The second inflatable balloon 405 is positioned adjacent to the opening 305 and ensures that the guide wire 15 is directed into the side branch vessel 30. Accordingly, other embodiments are within the scope of the following claims.

What is claimed is:

1. A system for delivering a guide wire to an artery and a side branch vessel of the artery, the system comprising:
a catheter defining a first lumen with a first opening and a second lumen with a second opening;
a first guide wire configured to extend through the first lumen; and
a second guide wire configured to extend through the second lumen, wherein the first opening is configured to direct the first guide wire into the side branch vessel, and the second opening is configured to direct the second guide wire into the artery.

2. The system of claim 1, wherein the catheter further comprises a radiopaque indicator adjacent to the first opening.

3. The system of claim 2, wherein the radiopaque indicator comprises at least one stud attached to the shaft.

4. The system of claim 2, wherein the radiopaque indicator comprises a ring that encircles at least a part of the circumference of the shaft.

5. The system of claim 4, wherein the ring includes an opening and the opening is adjacent to the first opening in the shaft.

6. The system of claim 4, wherein the ring includes an opening and the opening is coaxial with the first opening in the shaft.

7. The system of claim 4, wherein the ring includes an extension and the extension projects into the first opening in the shaft.

8. The system of claim 4, wherein the ring comprises a pair of bands that are joined at a first point of the circumference of the bands and are separated at a second point of the circumference of the bands, and the first opening is positioned between a separation at the second point of the pair of bands.

9. The system of claim 4, wherein the ring includes a cut-out section.

10. The system of claim 1, wherein the catheter comprises a first section including the first lumen and the second lumen and extending between the first end and the first opening, and a second section including the second lumen and extending between the first opening and the second end, and the second section has a smaller cross-section than a cross-section of the first section.

11. The system of claim 10, wherein the catheter includes an aimer positioned at least in part on the second section in a position that is adjacent to the first section.

12. The system of claim 11, wherein the aimer includes a surface that is configured to direct the guide wire in the first lumen in a direction away from the catheter when the guide wire passes through the first opening.

13. The system of claim 1, wherein the catheter includes an inflatable balloon positioned on the catheter adjacent to the first opening, whereby a guide wire passing through the first opening is deflected by the balloon.

14. The system of claim 1, further comprising at least one balloon inflation catheter configured to be delivered over one of the guide wires.

15. A delivery catheter configured to deliver a first guide wire to an artery and a second guide wire to a side branch vessel of the artery, the delivery catheter comprising:
a shaft having a first end and a second end;
a first lumen in the shaft passing to a first opening in the shaft; and
a second lumen passing to a second opening in the shaft.

16. The delivery catheter of claim 15, wherein the catheter further comprises a radiopaque indicator attached to the shaft at a location adjacent to the first opening.

17. The delivery catheter of claim 16, wherein the radiopaque indicator comprises at least one stud attached to the shaft.

18. The delivery catheter of claim 16, wherein the radiopaque indicator comprises a ring that encircles at least a part of the circumference of the shaft.

19. The delivery catheter of claim 18, wherein the ring includes an opening and the opening is adjacent to the first opening in the shaft.

20. The delivery catheter of claim 18, wherein the ring includes an opening and the opening is coaxial with the first opening in the shaft.

21. The delivery catheter of claim 18, wherein the ring includes an extension and the extension projects into the first opening in the shaft.

22. The delivery catheter of claim 18, wherein the ring comprises a pair of bands that are joined at a first point of the circumference of the bands and are separated at a second point of the circumference of the bands, and the first opening is positioned between separation at the second point of the pair of bands.

23. The delivery catheter of claim 18, wherein the ring includes a cut-out section.

24. The delivery catheter of claim 15, wherein the shaft comprises a first section including the first lumen and the second lumen and extending between the first end and the first opening, and a second section including the second lumen and extending between the first opening and the second end, and the second section has a smaller cross-section than a cross-section of the first section.

25. The delivery catheter of claim 24, wherein the catheter includes an aimer positioned at least in part on the second section in a position that is adjacent to the first section.

26. The delivery catheter of claim 25, wherein the aimer includes a surface that is configured to direct the guide wire in the first lumen in a direction away from the catheter when the guide wire passes through the first opening.

27. The delivery catheter of claim 15, wherein the catheter includes an inflatable balloon positioned on the catheter adjacent to the first opening, whereby a guide wire passing through the first opening is deflected by the balloon.

28. A method of catheterizing an artery and a side branch of the artery, the method comprising:
providing a side branch delivery catheter comprising a shaft having a first end and a second end, a first lumen in the shaft passing to a first opening in the shaft, and a second lumen passing to a second opening in the shaft;
inserting the delivery catheter into the vasculature;
advancing the delivery catheter into an artery;
positioning the first opening adjacent to the side branch of the artery;
inserting a first guide wire into the opening in the hub and advancing the guide wire into the first lumen;
inserting a second guide wire into the opening in the hub and advancing the guide wire into the second lumen; and
advancing the first guide wire through the first opening and into the side branch of the artery.

29. The method of claim 28, further comprising advancing the second guide wire through the second opening and into the artery.

30. The method of claim 28, further comprising:
providing an inflatable balloon catheter having an inflatable balloon;
advancing the inflatable balloon catheter over the first guide wire; and
deploying the inflatable balloon in the side branch of the artery.

31. The method of claim 28, further comprising:
providing an inflatable balloon catheter having an inflatable balloon;
advancing the inflatable balloon catheter over the second guide wire; and
deploying the inflatable balloon in the artery.

32. The method of claim 28, wherein positioning the first opening adjacent to the side branch of the artery comprises viewing a radiopaque indicator mounted on the catheter under fluoroscopy to orient the indicator relative to the side branch of the artery.
